(19) United States
(12) Patent Application Publication
(10) Pub. No.: US 2026/0272817 A1
(43) Pub. Date: Sep. 17, 2026

(54) SILICONE-FREE AQUEOUS CONDITIONING SHAMPOO COMPOSITION

(71) Applicant: THE PROCTER & GAMBLE COMPANY, Cincinnati, OH (US)

(72) Inventors: Dariush HOSSEINPOUR, Mason, OH (US); Kevin Lee DOYLE, Fairfield, OH (US); Mark Anthony BROWN, Union, KY (US)

(21) Appl. No.: 19/559,084

(22) Filed: Mar. 6, 2026

Related U.S. Application Data
(60) Provisional application No. 63/770,475, filed on Mar. 12, 2025.

(51) Int. Cl. A61K 8/92; A61K 8/44; A61K 8/46; A61K 8/81; A61Q 5/02; A61Q 5/12

(52) U.S. Cl. CPC A61K 8/922; A61K 8/44; A61K 8/463; A61K 8/8129; A61Q 5/02; A61Q 5/12; A61K 2800/33; A61K 2800/34

(57) ABSTRACT

An aqueous conditioning shampoo composition comprises a detersive surfactant, botanical oil, and about 0.02 wt. % to about 5 wt. % polyvinyl alcohol. The aqueous conditioning shampoo composition can be substantially free of silicone. The aqueous conditioning shampoo composition can have similar performance characteristics to a conditioning shampoo that contains silicone.

### CROSS REFERENCE TO RELATED APPLICATIONS

This application claims the benefit, under 35 U.S.C. § 119(e), to U.S. Provisional Application No. 63/770,475 filed Mar. 12, 2025, the entire disclosure of which is fully incorporated by reference herein.

### FIELD OF THE INVENTION

The present invention generally relates to conditioning shampoo compositions that are free of silicone, and more specifically to aqueous conditioning shampoo compositions that contain polyvinyl alcohol, a detersive surfactant, and botanical oil that provide similar conditioning performance as compared to shampoos that contain silicone.

### BACKGROUND OF THE INVENTION

Human hair becomes soiled due to its contact with the surrounding environment and from the sebum secreted by the scalp, in addition to the use of third-step stylers and hair treatments that leave a visually or tactilely noticeable residue behind on the hair. The soiling of hair causes it to have a dirty feel and an unattractive appearance, necessitating regular shampooing.

Shampooing cleans the hair by removing excess soil and sebum. However, shampooing can leave the hair in a wet, tangled, and generally unmanageable state. Once the hair dries, it is often left in a dry, rough, lusterless, and/or frizzy condition due to removal of the hair's natural oils. Therefore, shampoos that provide a cleansing and conditioning benefit to hair (hereinafter “conditioning shampoos”) are popular. Conditioning shampoos frequently contain silicones that coat the hair shaft, thereby locking in moisture, which reduces frizz and gives hair a coveted soft and silky feel. Silicones can provide these conditioning benefits while not interfering with cleansing efficacy.

However, some consumers prefer hair care products that are silicone-free. Many alternatives, such as natural oils, have been incorporated into shampoo compositions. However, these alternatives are generally less effective and/or leave the hair looking and feeling greasy and/or dirty.

Therefore, there is a need for a silicone-free shampoo composition that provides good conditioning and cleaning benefits.

### SUMMARY OF THE INVENTION

An aqueous conditioning shampoo composition comprising: (a) a detersive surfactant; (b) botanical oil; and (c) about 0.02 wt. % to about 5 wt. % polyvinyl alcohol. The composition is substantially free of silicone.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 is a bar graph showing the average detangling force in gram force (gf) values for wet hair in Example 1;

FIG. 2 is a bar graph showing the average detangling force in gram force (gf) values for dry hair in Example 1;

FIG. 3 is a table having shading showing the concentrations of fatty alcohol delivered to hair in Example 2; and

FIG. 4 is a table having shading showing viscosity as a function of the polyvinyl alcohol viscosity grades and degrees of hydrolysis for Example 3.

### DETAILED DESCRIPTION OF THE INVENTION

Conditioning shampoo compositions comprising various combinations of detersive surfactant and hair conditioning agents are known. Generally, these compositions comprise an anionic surfactant, cationic polymer, and silicone that combine to form coacervate, which deposits on hair during the wash, ultimately giving the hair a smooth texture and clean, silky feel.

However, some consumers want a liquid conditioning shampoo that is silicone-free. It is difficult to formulate a consumer-acceptable conditioning shampoo that is silicone-free because it is a very effective hair conditioning agent, making it difficult to find a composition that matches its performance particularly the smooth, clean feeling of dry hair.

Generally, conditioning shampoo compositions need to provide benefits in several key areas to be considered acceptable for consumers. These areas include viscosity and polymer deposition, among others. Polymer deposition is related to performance in wet detangling, which related to the ease of wet combing and dry smoothness related to the ease of dry combing, both of which are tied to smooth, clean feeling dry hair. While initially appealing, replacing silicone with alternative conditioning agents, such as botanical oils, can be difficult as it is difficult to provide compositions which deliver consumer-acceptable performance in all key areas. For example, botanical oils can negatively impact product viscosity by disrupting the salt-thickening mechanism of the micellar-based surfactant system. Moreover, compositions including botanical oil generally can still struggle to provide performance related to smooth, clean feeling dry hair. Indeed, inclusion of botanical oils to match silicone often leaves hair that looks and/or feels dirty, oily, and/or greasy.

It was found that an aqueous conditioning shampoo composition that contains detersive surfactant, botanical oil, and about 0.02 wt. % to about 5 wt. % of polyvinyl alcohol (PVOH) can meet or exceed the performance of conditioning shampoo comprising silicone. In embodiments, the botanical oil, included in the formulation with PVOH, comprises triglycerides and esters of fatty acids, wherein the fatty acids are either saturated, monounsaturated, or polyunsaturated and wherein the fatty acids contain varying chain lengths ranging from C8 to C30. In embodiments, the botanical oil, included in the formulation with PVOH, comprises one or more of soybean oil, canola oil, safflower oil, argan oil, jojoba oil, coconut oil, shea butter, orange peel wax, tea tree oil, and rice bran oil. The botanical oil may comprise about 0.2 wt. % to about 5 wt. % of the total composition.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All such percentages or weights as they pertain to listed ingredients are based on the active level and, therefore, do not include carriers or by-products that may be included in commercially available materials. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under about 1 atmosphere of pressure and at about 50% relative humidity. All ranges are inclusive and combinable. For example, all numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range of plus or minus 20% or less of the stated value (e.g., plus or minus 15% or less, 10% or less, 5% or less, or even 3% or less).

“Apply” or “application,” as used in reference to a composition, means to apply or spread the composition onto a human keratinous surface such as the skin or hair.

“Charge density” (“CD”) means the ratio of positive charges on a polymer to the molecular weight of the polymer.

“Co-surfactant” refers to a surfactant added to a cleansing composition to help solubilize the detersive surfactant and/or other materials in the composition, enhance lather volume, modify lather texture, and/or modify the viscosity of the composition.

“Detersive surfactant” refers to a surfactant added to a cleansing composition to provide the primary cleansing benefit of removing dirt, oil, and other contaminants from skin and/or hair.

“Molecular weight” or “Molecular weight” refers to the weight average molecular weight unless otherwise stated. Molecular weight is measured using industry standard method, gel permeation chromatography (“GPC”).

“Polymer,” as used herein, includes materials whether made by polymerization of one type of monomer or made by two (i.e., copolymers) or more types of monomers.

“Silicone-free” or “free of silicone” means that the composition contains no detectable silicone or silicone containing compounds.

“Substantially free of” means a composition or ingredient comprises 3% or less (e.g., 1% or less, 0.5% or less, 0.25% or less) of a subject material, by weight of the composition or ingredient. The term “substantially free” as used herein may also mean that the specific material is not added to the composition but may still be present in small quantities in a raw material that is included in the composition.

“Suitable for application to human hair,” as used herein, means that the compositions or components thereof so described are acceptable for use in contact with human hair and the scalp and skin without undue toxicity, incompatibility, instability, allergic response, and the like.

“Sulfated surfactant” refers to a surfactant that contains a sulfate moiety. Some non-limiting examples of sulfated surfactants are sodium lauryl sulfate, sodium laureth sulfate, ammonium lauryl sulfate, and ammonium laureth sulfate.

“Surfactant” refers to a substance consisting of molecules that have both hydrophilic (water-attracting) and hydrophobic (water-repelling) parts and which lowers the surface tension of the medium in which it is dissolved, and/or the interfacial tension with other phases, and, accordingly, is positively adsorbed at the liquid/vapor and/or at other interfaces.

“Water soluble,” as used herein, means that the material is soluble in water in the present composition. In general, the material should be soluble at 25° C. at a concentration of 0.1% by weight of the water solvent, alternatively at 1%, alternatively at 5%, and alternatively at 15%.

### Conditioning Shampoo Composition

The aqueous conditioning shampoo compositions herein comprise a detersive surfactant, a botanical oil, and polyvinyl alcohol. In some instances, the aqueous conditioning shampoo composition may include a co-surfactant, for example, to help solubilize the detersive surfactant or another ingredient in the composition. In some instances, the botanical oil may comprise triglycerides and esters of fatty acids, wherein the fatty acids are either saturated, monounsaturated, or polyunsaturated and contain varying chain lengths ranging from C8 to C30. In still further instances, the botanical oil may comprise soybean oil, canola oil, safflower oil, argan oil, jojoba oil, coconut oil, shea butter, orange peel wax, tea tree oil, and rice bran oil. The conditioning shampoo composition may also include a cationic conditioning polymer to aid in the appearance and/or feel of the hair.

### Botanical Oils

The aqueous conditioning shampoo composition herein includes botanical oils. Botanical oils of the type described herein typically are composed of triglycerides and esters of fatty acids. These fatty acids may be either saturated, monounsaturated or polyunsaturated and contain varying chain lengths ranging from C8 to C30. Exemplary fatty acids include saturated fatty acids such as lauric acid (dodecanoic acid), myristic acid (tetradecanoic acid), palmitic acid (hexadecanoic acid), stearic acid (octadecanoic acid), arachidic acid (eicosanoic acid), and lignoceric acid (tetracosanoic acid); unsaturated acids include such fatty acids as palmitoleic (a C16 acid), and oleic acid (a C18 acid); polyunsaturated acids include such fatty acids as linoleic acid (a di-unsaturated C18 acid), linolenic acid (a tri-unsaturated C18 acid), and arachidonic acid (a tetra-unsubstituted C20 acid).

Botanical oils further comprise esters of fatty acids in random placement onto the three sites of the trifunctional glycerine molecule. Different botanical oils have different ratios of fatty acids, and within a given botanical oil there is a range of acids, as well, depending on factors such as where a vegetable or crop is grown, maturity of the vegetable or crop, the weather during the growing season, etc. Thus, it is difficult to have a specific or unique structure for any given botanical oil, but rather a structure is typically based on a statistical average. For example, soybean oil contains a mixture of stearic acid, oleic acid, linoleic acid, and linolenic acid in the ratio of 15:24:50:11, and an average number of double bonds of 4.4-4.7 per triglyceride. One method of quantifying the number of double bonds is the iodine value (IV) which is defined as the number of grams of iodine that will react with 100 grams of oil. Therefore, for soybean oil, the average iodine value range is from 120-140. Soybean oil may comprise about 95% by weight or greater (e.g., 99% weight or greater) triglycerides of fatty acids. Major fatty acids in the polyol esters of soybean oil include saturated fatty acids, as a non-limiting example, palmitic acid (hexadecanoic acid) and stearic acid (octadecanoic acid), and unsaturated fatty acids, as a non-limiting example, oleic acid (9-octadecenoic acid), linoleic acid (9,12octadecadienoic acid), and linolenic acid (9,12,15-octadecatrienoic acid).

Exemplary botanical oils include, but are not limited to, canola oil, safflower oil, argan oil, jojoba oil, coconut oil, shea butter, orange peel wax, tea tree oil, and rice bran oil.

The composition may include 0.2 wt. %, 0.3 wt. % to 5.0 wt. % of the botanical oil, alternatively 0.3 wt. % to 3 wt. % botanical oil, alternatively 0.4 wt. % to 1.5 wt. % botanical oil, alternatively 0.5 wt. % to 1 wt. % botanical oil, alternatively 0.6 wt. % to 0.9 wt. % botanical oil, and alternatively 0.2 wt. % to 0.5 wt. % botanical oil, by weight of the total composition.

### Polyvinyl Alcohol

The aqueous conditioning shampoo composition herein includes polyvinyl alcohol (PVOH). The composition may include 0.02 wt. % to 5.0 wt. % PVOH, alternatively 0.05 wt. % to 2 wt. % PVOH, alternatively 0.1 wt. % to 1.5 wt. % PVOH, alternatively 0.15 wt. % to 1 wt. % PVOH, alternatively 0.2 wt. % to 0.9 wt. % PVOH, and alternatively 0.2 wt. % to 0.5 wt. % PVOH, by weight of the total composition. The PVOH can have a degree of hydrolysis greater than 80%, alternatively greater than 88%, alternatively greater than 98%, and alternatively greater than 99%. The PVOH can have a viscosity within a range of 1 mPa-s to 100 mPa-s, and alternatively greater than 20 mPa-s as measured as a 4% aqueous solution at 20° C. The PVOH Viscosity is measured according to the PVOH Viscosity test method, described herein.

### Detersive Surfactants

The aqueous conditioning shampoo composition herein includes at least one detersive surfactant to provide cleansing performance. In aspects, the detersive surfactant is an anionic surfactant. The concentration of the one or more anionic surfactants in the shampoo composition can be sufficient to provide the desired cleaning and lather performance, and generally ranges from 4 wt. % to 25 wt. % alternatively 8 wt. % to 20 wt. % alternatively 8 wt. % to 15 wt. % and alternatively 10% to 14%, by weight of the composition.

Anionic surfactants suitable for use herein include alkyl and alkyl ether sulfates of the formula ROSO₃M and RO(C₂H₄O)ₓSO₃M, wherein R is alkyl or alkenyl of 8 to 18 carbon atoms, x is 1 to 10, and M is a water-soluble cation such as ammonium, sodium, potassium, and triethanolamine cation or salts of the divalent magnesium ion with two anionic surfactant anions. The alkyl ether sulfates may be made as condensation products of ethylene oxide and monohydric alcohols having 8 to 24 carbon atoms. The alcohols can be derived from fats such as coconut oil, palm oil, palm kernel oil, or tallow, or can be synthetic.

Other suitable anionic surfactants include water-soluble salts of the organic, sulfonic acids of the general formula [R¹—SO₃M]. R¹ being a straight chain aliphatic hydrocarbon radical having from 13 to 17 carbon atoms, alternatively from 13 to 15 carbon atoms. M being a water-soluble cation such as ammonium, sodium, potassium, and triethanolamine cation or salts of the divalent magnesium ion with two anionic surfactant anions. These materials are produced by the reaction of SO₂ and O₂ with suitable chain length normal paraffins (C₁₄-C₁₇) and are sold commercially as sodium paraffin sulfonates.

In one example, the anionic surfactant may be a combination of sodium lauryl sulfate and sodium laureth sulfate.

Examples of suitable anionic surfactants can include, but are not limited to, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium trideceth sulfate, sodium tridecyl sulfate, sodium methyl lauroyl taurate, sodium methyl cocoyl taurate, sodium lauroyl isethionate, sodium cocoyl isethionate, sodium laurethsulfosuccinate, sodium laurylsulfosuccinate, sodium tridecyl benzene sulfonate, sodium dodecyl benzene sulfonate, or mixtures thereof.

In some examples, the composition can include one or more sulfate-free anionic surfactants such as sodium, ammonium, or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

Some particularly suitable examples of sulfate-free anionic surfactants can include isethionates, sarcosinates, sulfonates, taurates, or mixtures thereof.

Suitable isethionate surfactants can include the reaction product of fatty acids esterified with isethionic acid and neutralized with sodium hydroxide. Suitable fatty acids for isethionate surfactants can be derived from coconut oil or palm kernel oil including amides of methyl tauride. Non-limiting examples of isethionates can be selected from the group consisting of sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate, sodium stearoyl methyl isethionate, or mixtures thereof.

An amino acid-based anionic surfactant can be a sarcosinate, for instance an acyl sarcosinate. Non-limiting examples of sarcosinates can be selected from the group consisting of sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroylglutamate/lauroylsarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium oleoyl sarcosinate, sodium palmitoyl sarcosinate, TEA-cocoyl sarcosinate, TEA-lauroyl sarcosinate, TEA-oleoyl sarcosinate, TEA-palm kernel sarcosinate, and combinations thereof.

Non-limiting examples of sulfonates can include alpha olefin sulfonates, alkyl olefin sulfonates, linear alkylbenzene sulfonates, sodium laurylglucosides hydroxypropylsulfonate, and combinations thereof.

### Co-Surfactants

The aqueous shampoo composition may include a co-surfactant selected from anionic surfactants, amphoteric surfactants, zwitterionic surfactants, non-ionic surfactants, and combinations of these. Some non-limiting examples of anionic surfactants include non-taurate, non-sulfate anionic surfactants such as isethionates, carboxylates, sulfonates (e.g., alpha olefin sulfonates, alkyl olefin sulfonates, linear alkylbenzene sulfonates, alkyl glyceryl sulfonates, sodium laurylglucosides hydroxypropylsulfonate), branched alkyl sulfates, sulfosuccinates, sulfoacetates, sulfolaurates, amino acid-based surfactants (e.g., glycinates, sarcosinates, alaninates, glutamates), lactate- and lactylate-based surfactants (e.g., sodium lauroyl lactate and sodium lauroyl lactalyte), phosphate ester surfactants and combinations thereof.

Some non-limiting examples of amphoteric and/or zwitterionic surfactants include derivatives of aliphatic secondary and tertiary amines in which one of the aliphatic substituents contains from 8 to 18 carbon atoms and one aliphatic substituent contains an anionic group such as a carboxy, sulfonate, phosphate, or phosphonate group. Zwitterionic surfactants are surfactants whose polar functional group has two permanent charges that do not change with changing pH. Amphoteric surfactants have polar functional groups whose charge depends on the pH of the solution and can exhibit different charges as the pH changes from acid to neutral to basic, ranging from cationic to zwitterionic and potentially even to anionic. Some non-limiting examples of zwitterionic surfactants include amidosulfobetaines, amidopropylbetaines, hydroxysultaines, amidopropyl hydroxysultaines, lauryl hydroxysultaine, coco-hydroxysultaine, and combinations thereof. Some non-limiting examples of amphoteric surfactants include amphoacetates, amphodiacetates, betaines (including alkyl betaines, coco-betaine, and lauryl betaine), amidobetaines (e.g., cocamidopropyl betaine and lauramidopropyl betaine), propionates, hydroxysultaines, and combinations thereof.

Some non-limiting examples of non-ionic surfactants include glyceryl esters of alkanoic acids, polyglyceryl esters of alkanoic acids, propylene glycol esters of alkanoic acids, sorbitol esters of alkanoic acids, alkanolamides, alkoxylated amides, alkyl glycosides, alkyl polyglucosides, decyl glucoside, lauryl glucoside, coco-glucoside, acyl glucamides, amine oxides and combinations thereof. Some particularly suitable examples of non-ionic surfactants include cocamide, cocamide MEA, cocamide methyl MEA (CMMEA), PPG-2 cocamide, PPG-2 hydroxyethyl cocamide, cocamide diethanolamine (DEA), PPG-2 hydroxyethyl isostearamide, lauroyl/myristoyl methyl glucamide, capryloyl/caproyl methyl glucamide, cocoyl methyl glucamide, decyl glucoside, coco-glucoside, lauryl glucoside, lauramine oxide, cocamine oxide and combinations thereof.

More specific examples of the optional co-surfactants described above are disclosed in US 2019/0105246, US 2018/0098923, U.S. Pat. No. 9,271,908, WO 2020/016097, and Mccutcheon's Emulsifiers and Detergents, 2019, MC Publishing Co.

The co-surfactant may be present in the shampoo compositions at 1 wt. % to 15 wt. % (e.g., 2 wt. % to 10 wt. %, 3 wt. % to 9 wt. %, 4 wt. % to 8 wt. %, or 5 wt. % to 7 wt. %). The amount of co-surfactant in the composition can be important and should be tailored to balance solubility and/or viscosity building with cleaning and/or conditioning benefits. For example, too much amphoteric co-surfactant can make the surfactant system less salt-tolerant and may impede the ability of the surfactant system to form a suitable coacervate upon dilution with water.

### Cationic Polymer

The aqueous conditioning shampoo compositions herein may include 0.05 wt. % to 3 wt. % of a cationic polymer (e.g., 0.1 wt. % to 2 wt. % or even 0.2 wt. % to 0.8 wt. % to provide an improved appearance, feel, or deposition benefits to hair or skin. The cationic polymer can have a weight average molecular weight of 50 kDa to 5 MDa (e.g., 500 kDa-4 MDa, 1-3 MDa, 1.2-2 MDa, or even 1.4-1.8 MDa) and a charge density of 0.2 meq/g to 12 meq/g (e.g., 0.4-10 meq/g, 0.4-5 meq/g, 0.4-4 meq/g, 0.4-3 meq/g, or even 0.4-2 meq/g). The charge densities can be measured at the pH of the intended use of the shampoo composition, which can be pH 3 to pH 9 (e.g., pH 4-8 or pH 4.5-6.5).

The cationic polymers may include cationic, nitrogen-containing moieties such as quaternary ammonium or cationic protonated amino moieties. The cationic protonated amines can be primary, secondary, or tertiary amines, depending upon the particular species and the selected pH of the composition. Anionic counterions can be used in association with the cationic polymers, as long as the polymers remain soluble. Examples of suitable counterions include halide counterions (e.g., chloride, fluoride, bromide, iodide).

Some non-limiting examples of cationic polymers include copolymers of vinyl monomers having cationic protonated amine or quaternary ammonium functionalities with water-soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone. Some non-limiting examples of cationic protonated amino and quaternary ammonium monomers include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Additional nonlimiting examples of cationic polymers include copolymers of 1-vinyl-2-pyrrolidone and 1-vinyl-3-methylimidazolium salt (e.g., chloride salt) (referred to in the industry by the Shampoo Products Council (“PCPC”) as Polyquaternium-16); copolymers of 1-vinyl-2-pyrrolidone and dimethylaminoethyl methacrylate (Polyquaternium-11); cationic diallyl quaternary ammonium-containing polymers, including, for example, dimethyldiallylammonium chloride homopolymer, copolymers of acrylamide and dimethyldiallylammonium chloride (Polyquaternium-6 and Polyquaternium-7, respectively); amphoteric copolymers of acrylic acid including copolymers of acrylic acid and dimethyldiallylammonium chloride (Polyquaternium-22), terpolymers of acrylic acid with dimethyldiallylammonium chloride and acrylamide (Polyquaternium-39), and terpolymers of acrylic acid with methacrylamidopropyl trimethylammonium chloride and methylacrylate (Polyquaternium-47). In some aspects, suitable cationic substituted monomers include cationic substituted dialkylaminoalkyl acrylamides, dialkylaminoalkyl methacrylamides, and combinations thereof. The cationic polymer can be AM:TRIQUAT which is a copolymer of acrylamide and 1,3-Propanediaminium, N-[2-[[[dimethyl[3-[(2-methyl-1-oxo-2-propenyl)amino]propyl]ammonio]acetyl]amino]ethyl]2-hydroxy-N,N,N′,N′,N′-pentamethyl-, trichloride (Polyquaternium-76). AM:TRIQUAT may have a charge density of 1.6 meq/g and a molecular weight of 1.1 MDa.

In some aspects, the cationic monomer can be polymethyacrylamidopropyl trimonium chloride, available under the trade name Polycare® 133, from Solvay (Brussels, Belgium). Copolymers of the cationic monomer may also be suitable, and the charge density of the total copolymer can be 2.0 meq/g to 4.5 meq/g.

Other cationic polymers include polysaccharide polymers, such as cationic cellulose derivatives and cationic starch derivatives. In certain embodiments, a cationic cellulose polymer can be selected from the salts of hydroxyethyl cellulose reacted with trimethyl ammonium substituted epoxide, referred to in the industry (PCPC) as Polyquaternium-10 and available from Dow Chemical Company as UCARE™ JR-30M, KG-30 M and LR-30M. Other examples of cationic cellulose polymers include polymeric quaternary ammonium salts of hydroxyethyl cellulose reacted with lauryl dimethyl ammonium-substituted epoxide referred to in the industry (PCPC) as Polyquaternium-24.

Further examples of cationic polymers include cationic guar gum derivatives, such as guar hydroxypropyltrimonium chloride, such as the Jaguar® series available from Solvay and the N-Hance™ and AquaCat™ series from Ashland (Wilmington, Delaware). Additional disclosure of cationic guar gum derivatives can be found in U.S. Patent No. U.S. Pat. No. 6,930,078.

In some instances, the cationic polymer may include a synthetic cationic polymer or derivative thereof present at 0.025 wt. % to about 5 wt. % Preferred synthetic cationic polymers are generally water-soluble or dispersible and non-crosslinked. In some instances, the synthetic cationic polymer can be a copolymer that includes one or more cationic monomer units and one or more nonionic or anionic monomer units, as long as the copolymer has a net positive charge. Synthetic cationic polymers can have a cationic charge density of 0.5 meq/g to 12 meg/g and an average molecular weight of 1 kDa to 5 MDa. Some non-limiting examples of synthetic cationic polymers are described in US 2003/0223951.

### Structurant Compounds

The aqueous conditioning shampoo composition may optionally include one or more glyceride ester compounds as structurant. Traditionally glyceride ester compounds may be used as a structurant for aqueous conditioning shampoo compositions. For example, Thixcin® R is trihydroxystearin, a commercial hydrogenated castor oil produced by Elementis Specialties of New Jersey, and marketed as a stabilizer and structurant for shampoo compositions. Suitable glyceride esters for shampoo compositions described herein can be selected from any crystallizable glyceride esters which can allow for the formation of a coacervate in shampoo compositions including a suitable surfactant and a cationic polymer. For example, suitable glyceride esters are hydrogenated castor oils such as trihydroxystearin or dihydroxystearin.

Examples of additional crystallizable glyceride esters can include the substantially pure triglyceride of 12-hydroxystearic acid. 12-hydroxystearic acid is the pure form of a fully hydrogenated triglyceride of 12-hydrox-9-cis-octadecenoic acid. As can be appreciated, many additional glyceride esters are possible. For example, variations in the hydrogenation process and natural variations in castor oil can enable the production of additional suitable glyceride esters from castor oil.

Suitable glyceride esters can also be formed from mixtures of one or more glycerides. For example, a mixture of glycerides including about 80% or more, by weight of the mixture, castor oil, can be suitable. Other suitable mixtures can include mixtures of only triglycerides, mixtures of diglycerides and triglycerides, mixtures of triglycerides with diglycerides and limited amounts, e.g., less than about 20%, by weight of the mixture, of monoglyerides; or any mixture thereof which includes about 20% or less, by weight of the mixture, of a corresponding acid hydrolysis product of any of the glycerides. About 80% or more, by weight of a mixture, can be chemically identical to a glyceride of fully hydrogenated ricinoleic acid, i.e., glyceride of 12-hydroxystearic acid. Hydrogenated castor oil can be modified such that in a given triglyceride, there will be two 12-hydroxystearic moieties and one stearic moiety. Alternatively, partial hydrogenation can be used. However, poly(oxyalkylated) castor oils are not suitable because they have unsuitable melting points.

Castor oils include glycerides, especially triglycerides, comprising C10 to C22 alkyl or alkenyl moieties which incorporate a hydroxyl group. Hydrogenation of castor oil produces hydrogenated castor oil by converting double bonds, which are present in the starting oil as ricinoleyl moieties. These moieties are converted to ricinoleyl moieties, which are saturated hydroxyalkyl moieties, e.g., hydroxystearyl. The hydrogenated castor oil (HCO) herein may, in some embodiments, be selected from: trihydroxystearin; dihydroxystearin; and mixtures thereof. The HCO may be processed in any suitable starting form, including, but not limited those selected from solid, molten and mixtures thereof. Useful HCO may have the following characteristics: a melting point of from about 40° C. to about 100° C., alternatively from about 65° C. to about 95° C.; and/or Iodine value ranges of from about 0 to about 5, alternatively from about 0 to about 4, and alternatively from about 0 to about 2.6. The melting point of HCO can measured using DSC: Differential Scanning calorimetry.

Suitable HCO include those that are commercially available. Non-limiting examples of commercially available HCO suitable for use include: THIXCIN-R® (supplied by Elementis), which is supplied as a powder having small particles (99 wt % smaller than of 44 micrometers). The HCO may be in the present shampoo composition from about 0.01% to about 4%.

The invention is not intended to be directed only to the use of hydrogenated castor oil. Any other suitable crystallizable glyceride may be used. In one example, the structurant is substantially pure triglyceride of 12-hydroxystearic acid. This molecule represents the pure form of a fully hydrogenated triglyceride of 12-hydrox-9-cis-octadecenoic acid. In nature, the composition of castor oil may vary somewhat. Likewise, hydrogenation procedures may vary. Any other suitable equivalent materials, such as mixtures of triglycerides wherein at least about 80 wt % is from castor oil, may be used. Exemplary equivalent materials comprise primarily, or consist of, triglycerides; or comprise primarily, or consist of, mixtures of diglycerides and triglycerides; or comprise primarily, or consist of, mixtures of triglyerides with diglycerides and limited amounts, e.g., less than about 20 wt % of the glyceride mixtures, of monoglyerides; or comprise primarily, or consist of, any of the foregoing glycerides with limited amounts, e.g., less than about 20 wt %, of the corresponding acid hydrolysis product of any of said glycerides.

### Liquid Carrier

The aqueous conditioning shampoo composition may optionally include 20-95% of an aqueous carrier such as water and/or a water-miscible solvent. The type and amount of aqueous carrier should be selected to provide the composition with the desired rheological properties. The liquid carrier can be water with, e.g., less than 10 wt. %, 7 wt. %, 5 wt. %, 3 wt. %, 1 wt. %, 0.5 wt. % or even 0 wt. % miscible organic solvent. Some nonlimiting examples of organic solvents include lower alkyl alcohols (e.g., ethanol and isopropanol) and polyhydric alcohols (e.g., propylene glycol, hexylene glycol, glycerin, and propane diol). A shampoo composition can include ≥50%, alternatively ≥60%, alternatively ≥70%, alternatively ≥75%, by weight, of a liquid carrier.

### Optional Ingredients

The aqueous conditioning shampoo compositions described herein may include a variety of optional ingredients to tailor the properties and characteristics of the composition, as desired. The optional ingredients may be materials that are commonly included in compositions of the type. The optional ingredients should be physically and chemically compatible with the essential components of the shampoo composition and should not otherwise unduly impair the stability, aesthetics, or performance of the composition. Individual concentrations of optional components can generally range from 0.001% to 10%.

Some non-limiting examples of optional ingredients that can be included in the aqueous conditioning shampoo compositions herein include deposition aids, cationic polymers, conditioning agents (including gel network, triglyceride oils, hydrocarbon oils, fatty esters, silicones), anti-dandruff agents (e.g., zinc pyrithione, zinc carbonate, piroctone olamine, piroctone, ciclopirox, rilopirox, MEA-Hydroxyoctyloxypyridinone, azoxystrobin, sulfur, azoles, salicylic acid and selenium sulfide, 1,10-phenanthroline), anti-microbial agents, suspending agents, viscosity modifiers, dyes, pigments, nonvolatile solvents or diluents (water soluble and insoluble), pearlescent aids, foam boosters, pediculocides, pH adjusting agents, perfumes, preservatives, chelants, proteins, vitamins, amino acids, skin active agents, sunscreens, UV absorbers, stabilizers, and combinations of these.

### Dispersed Gel Network

The aqueous conditioning shampoo composition may include a dispersed gel network phase to provide a suitable cleaning benefit to the composition in combination with the anionic surfactant. The shampoo composition can contain 1 wt. % to 12 wt. % dispersed gel network phase, alternatively 1 wt. % to 7 wt. % dispersed gel network phase, alternatively 1.25 wt. % to 6 wt. %, alternatively 1.5 wt. % to 5 wt. %, and alternatively 2 wt. % to 4 wt. %.

Suitable dispersed gel networks can be formed by combining a fatty alcohol and a gel network surfactant in a suitable ratio and heating the dispersion to a temperature above the melting point of the fatty alcohol. Additional details of suitable gel networks are described in G.M. Eccleston, “Functions of Mixed Emulsifiers and Emulsifying Waxes in Dermatological Lotions and Creams,” Colloids and Surfaces A: Physiochem, and Eng. Aspects 123-124 (1997) 169-182; and by G. M Eccleston, “The Microstructure of Semisolid Creams”, Pharmacy International, Vol. 7, 63-70 (1986), each of which is incorporated by reference herein.

In some aspects, it may be desirable to pre-form the gel network phase, e.g., a gel network premix, which means that at least fifty percent of the mixture of the fatty alcohol, gel network surfactant, and liquid carrier are in a substantially solid crystalline phase prior to addition to the other components of the shampoo composition. When the dispersed gel network is pre-formed, the gel network component can be prepared as a separate pre-mix, which, after being cooled, can be subsequently incorporated with an anionic surfactant and any other components of a shampoo composition. While not intending to be limited by theory, it is believed that incorporation of a pre-formed gel network component with the anionic surfactant and other components of the shampoo composition allows the formation of a substantially equilibrated lamellar dispersion (“ELD”) in the final composition.

The ELD is a dispersed lamellar or vesicular phase resulting from the pre-formed gel network component substantially equilibrating with the anionic surfactants, carrier, and other optional components of a shampoo composition. This equilibration occurs upon incorporation of the pre-formed gel network component with the other components of a shampoo composition and can be effectively completed within 24 hours after incorporation.

### Gel Network Fatty Alcohol

The dispersed gel network phase may include a fatty alcohol (e.g., C10-C40 fatty alcohols) at 0.05 wt. % or more by weight of the composition (e.g., 0.05 wt. % to 25 wt. %, 0.5 wt. % to 20 wt. %, or 1 wt. % to 8 wt. %). The fatty alcohol may be a straight or branched chain and can be saturated or unsaturated. As can be appreciated, suitable fatty alcohols can be of natural, vegetable, or synthetic origin. In some aspects, it may be desirable to mix several fatty alcohols to provide a dispersed gel network phase with a melt transition temperature of 38° C. or greater such as, for example, a mixture of cetyl alcohol and stearyl alcohol at a ratio of between 20:80 and 80:20. Some non-limiting examples of fatty alcohols that may be suitable for use herein include cetyl alcohol, stearyl alcohol, arachidyl alcohol, behenyl alcohol, C21 fatty alcohol (1-heneicosanol), C23 fatty alcohol (1-tricosanol), C24 fatty alcohol (lignoceryl alcohol, 1-tetracosanol), C26 fatty alcohol (1-hexacosanol), C28 fatty alcohol (1-octacosanol), C30 fatty alcohol (1-triacontanol), C20-40 alcohols (e.g., Performacol® 350 and 425 Alcohols, available from New Phase Technologies), C30-50 alcohols (e.g., Performacol® 550 Alcohol), C40-60 alcohols (e.g., Performacol® 700 Alcohol), or mixtures thereof.

The aqueous conditioning shampoo compositions may include fatty alcohol and at least a portion, if not most of the fatty alcohol is part of the dispersed gel network phase. The shampoo composition can include a fatty acid in an amount of least 1 wt. %, alternatively 1 wt. % to 8 wt. %, alternatively 1.25 wt. % to 6 wt. %, alternatively 1.5 wt. % to 5 wt. %, alternatively 2 wt. % to 5 wt. %, and alternatively 2 wt. % to 4 wt. %.

In one example, the weight ratio of the fatty alcohol to the gel network surfactant in the gel network component may be greater than 1:9, alternatively 1:5 to 100:1, and alternatively 1:1 to 50:1.

### Gel Network Surfactant

The aqueous conditioning shampoo compositions may include a gel network surfactant. The composition may include a gel network surfactant at 0.01 wt. % to 15 wt. %, alternatively 0.1 wt. % to 10 wt. %, and alternatively 0.2 wt. % to 5 wt. %.

The gel network surfactant can be any suitable anionic, zwitterionic, amphoteric, cationic, nonionic surfactant, or mixtures thereof, as described herein. In some examples, the gel network surfactant is free of or substantially free of sulfates. The anionic surfactant and the gel network surfactant can be independently selected and can be the same or different. In some aspects, the gel network surfactant has a hydrophobic tail group with a chain length of 10 to 40 carbon atoms. The hydrophobic tail group may be alkyl, alkenyl (containing up to 3 double bonds), alkyl aromatic, or branched alkyl. Mixtures of more than one gel network surfactant can also be used. Some non-limiting examples of gel network surfactants are disclosed in US 2006/0024256.

### Gel Network Water or Suitable Solvent

The dispersed gel network phase may also include water or suitable solvents. The water or suitable solvent and the gel network surfactant together contribute to the swelling of the fatty alcohol. This, in turn, leads to the formation and stability of the gel network. As used herein, the term “suitable solvent” refers to any solvent that can be used in place of or in combination with water in the formation of the gel network.

The aqueous conditioning shampoo compositions may comprise water or suitable solvents as part of the pre-formed dispersed gel network component in an amount suitable to achieve a gel network when combined with fatty alcohol and gel network surfactant according to the present invention. The gel network water or suitable solvent may be present in an amount of at least 0.05%, by weight, of the shampoo composition.

### Method of Making a Shampoo Composition

The aqueous conditioning shampoo compositions described herein are generally prepared by conventional methods such as those known in the art of making the compositions. Such methods typically involve mixing the ingredients in one or more steps to a relatively uniform state, with or without heating, cooling, application of vacuum, and the like. The compositions are prepared to optimize stability (physical stability, chemical stability, photostability) and/or delivery of the active materials.

### Method of Use

The shampoo compositions described herein can be used in a conventional manner for cleansing and conditioning of hair. Effective amounts of the composition for use generally range from 1 g to 50 g (e.g., 1 g to about 20 g). Generally, a method of treating hair can include applying the shampoo composition to the hair. For example, an effective amount of the shampoo composition can be applied to the hair, that has been wetted with water, and then the composition can be rinsed off. Application to the hair typically includes working the composition through the hair such that most or all the hair is contacted with the composition. The shampoo composition can be used as a liquid. In some aspects, the method for treating the hair or skin can include the steps of (a) wetting the hair or skin with water; (b) applying an effective amount of the shampoo composition to the hair, and (c) rinsing the applied areas of hair with water. These steps can be repeated as many times as desired to achieve the desired cleansing and conditioning benefit.

### Test Methods

### Cone/Plate Viscosity Measurement Method

The viscosities of the examples are measured by a Cone/Plate Controlled Stress Brookfield Rheometer R/S Plus, by Brookfield Engineering Laboratories, Stoughton, MA. The cone used (Spindle C-75-1) has a diameter of 75 mm and 1° angle. The liquid viscosity is determined using a steady state flow experiment at constant shear rate of 2 s-1 and at temperature of 26.7° C. The sample size is 2.5 ml to 3 ml and the total measurement reading time is 3 minutes.

### PVOH Viscosity Method

The viscosity of the PVOH is measured as a 4% aqueous solution by a Brookfield DV-II+pro cup and bob viscometer with ultra-low viscosity adaptor. The measurement geometry is cylindrical type 0 rotor. The sample temperature is 20° C. The shear rate is adjusted to accommodate PVOH variants with different viscosities while maintaining a torque within the range of 60-80%.

### Hair Texture Analysis Method

### Hair Substrate

Round ponytail hair tresses of general population, Chinese hair measuring 4 grams in weight and 8 inches in length were purchased from International Hair Importers & Products Inc., 87-29 Myrtle Ave., Glendale, NY 11385 and used in the hair treatment procedure that follows. Each test product was applied to 3 separate hair tresses (n=3).

### Hair Treatment

0.4 grams (0.1 g/gram hair) of a conditioning shampoo (e.g., Composition A) was applied to a hair tress that was first prewetted with 100-105 OF water and then squeegeed to remove any excess water. The product was applied down the hair tress in a zig-zag pattern, equally to the front and back of the hair tress (0.2 grams per side). The hair tress was then brushed, using a Goody Brush with large, stiff, plastic bristles, alternating down the front and back for 30 strokes at approximately 1 stroke per second for a total of 30 seconds. The tress was then rinsed using 100-105° F. water for 30 seconds while milking the switch at approximately 1 stroke per second. The tress was squeezed to remove any excess water. The test product was then reapplied to the hair tress a second time following the same procedure described above and then rinsed from the hair tress.

### Measurement of the Detangling Force

The detangling force required to comb a hair tress after treatment (wet and dry) was measured using the Texture Analyzer TA-XT Plus (manufactured by Stable Micro Systems), Instron 5542 (manufactured by Instron) or equivalent force measurement device. This method is an industry standard method for measuring wet/dry hair detangling forces related to combing, disclosed by TRI Princeton.

The hair tress is placed within the holder of the Texture Analyzer, fixed at the root end of the hair tress. The hair tress is positioned within the combs in series and then pulled through the combs by the Texture Analyzer, while the average force to pull the tress through each comb is recorded (=1 combing stroke). The hair tress is disengaged from the combs and returned to its pre-combing position. The hair tress is then combed 9 more times using the same combing procedure. The measured values represent average gram force (gf) values for combing strokes 2 through 6 of a 10-stroke test. Lower values represent less detangling force during combing and higher conditioning, which is desired.

### Fatty Alcohol Quantitation

Treated hair samples, each of which received five cycles of hair treatment according to the method above, are equilibrated. For each sample, approximately 0.1 g of hair is cut into 20-40 mm segments and placed into vials (n=4).

First, the hair is extracted gently with hexane, a nonpolar solvent, to remove the external oil. The hexane extraction consists of extracting the hair with hexane two times in a vortex mixer for 5 minutes each to dissolve and remove external oil, oils present on the hair's surface and then concentrating the dried residue, left after the hexane evaporates, in a second solvent, a 2:1 mixture of chloroform:methanol with 10 mM dimethylhexylamine (DMHA) and 0.33% formic acid. Thereafter, an aliquot, a small measured portion, of the external oil mixture is analyzed by Gas Chromatography (GC) to determine the external oil portion.

Next, the internal oil is extracted once using the second solvent and twice using a third solvent, a 1:1 mixture of chloroform:methanol with 15 mM DMHA and 0.5% formic acid to draw out oils within the hair. Each extraction is heated for 30 minutes at 65° C. with the hair to ensure thorough extraction and then combined and the dried residue, left after the hexane evaporates, is re-dissolved in the second solvent. Thereafter, an aliquot of the internal oil mixture is analyzed by GC to determine the external oil portion.

The oil portions are quantified by gas chromatography with flame ionization detection using a polydimethylsiloxane capillary column (2.5 m×0.240 mm×0.24 μm) with hydrogen mobile phase. Derivatized nonadecanoic acid and eicosanoic acid are used as internal standards.

### EXAMPLES

The following data and examples are provided to help illustrate the aqueous conditioning shampoo compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified. The levels given reflect the weight percent of the active material, unless otherwise specified.

### Example 1: Conditioning and Detangling Evaluation

The conditioning and detangling effect of ten compositions was evaluated. Table 1 provides composition and detangling force data for Examples 1-4 and Comparative Examples C1, C2, C3, C4, C5, and C6. The compositions for Examples 1-4 and C1, C2, C3, C4, C5 and C6 were relatively similar. For example, all ten compositions used the same amount of sodium laureth sulfate, sodium lauryl sulfate, cocamidoptopyl betaine, guar hydroxypropyl trimonium chloride, gel network premix, and hydrogenated castor oil. The exemplary compositions varied in the presence and amount of rice bran oil, PVOH (PVOH 28-98), associative thickener (ethylene glycol distearate) and a silicone-containing component (dimethicone). In general, the Comparative Examples (C1-C6) were missing at least one of rice bran oil and PVOH and Examples 1-4 all contained both a combination of both rice bran oil and PVOH.

C1 included no rice bran oil, PVOH, associative thickener, and silicone-containing component. C2 included no rice bran oil, associative thickener, and silicone-containing component but included 0.2% PVOH. C3 included no rice bran oil, associative thickener, and silicone-containing component but included 0.5% PVOH. C4 included no PVOH, associative thickener, and silicone-containing component but included 0.5% rice bran oil. C5 included no PVOH, associative thickener, and silicone-containing component but included 1% rice bran oil. C6 included no PVOH, and rice bran oil but included 1.5% associative thickener and 3% silicone-containing component. Thereby, C6 provided a silicone containing exemplary composition which served as a benchmark for the other exemplary compositions, which did not have a silicone-containing component.

Example 1 included 0.5% rice bran oil and 0.2% PVOH but no associative thickener and silicone-containing component. Example 2 included 0.5% rice bran oil and 0.5% PVOH but no associative thickener and silicone-containing component. Example 3 included 1% rice bran oil and 0.2% PVOH but no associative thickener and silicone-containing component. Example 4 included 1% rice bran oil and 0.5% PVOH but no associative thickener and silicone-containing component.

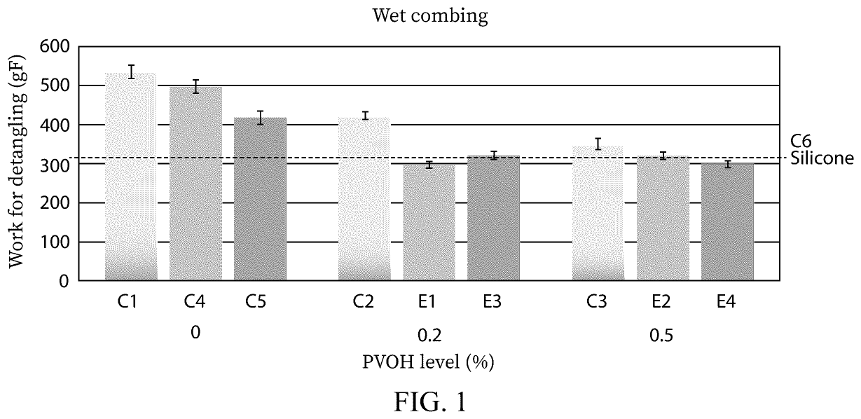

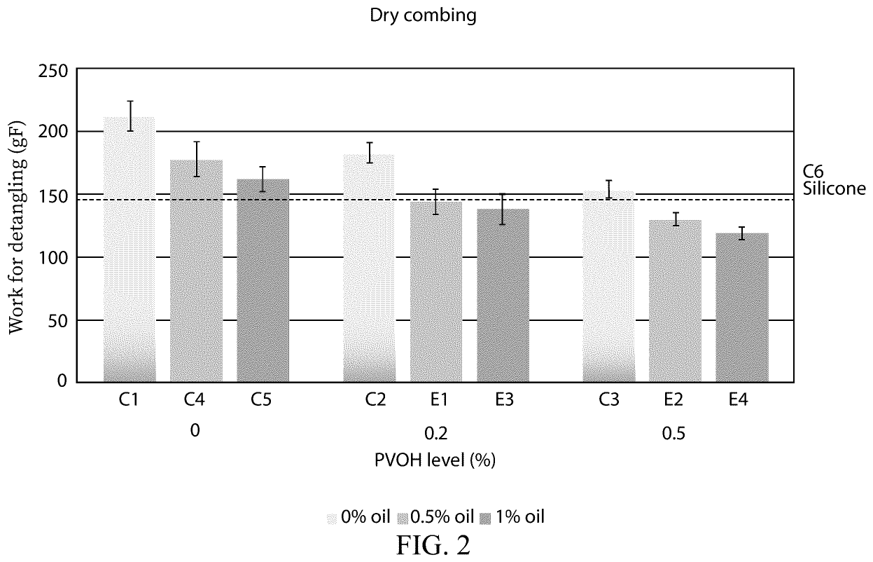

The detangling force was measured according to the methods described herein. The detangling force results are illustrated in FIG. 1 and FIG. 2. FIG. 1 shows results for wet hair and FIG. 2 shows results for dry hair.

TABLE 1

| | C1 | C2 | C3 | C4 | Ex. 1 | Ex. 2 | C5 | Ex. 3 | Ex. 4 | C6 |
|---|---|---|---|---|---|---|---|---|---|---|
| Sodium Laureth | 11.6 | 11.6 | 11.6 | 11.6 | 11.6 | 11.6 | 11.6 | 11.6 | 11.6 | 11.6 |
| Sulfate | | | | | | | | | | |
| Sodium Lauryl | 1.4 | 1.4 | 1.4 | 1.4 | 1.4 | 1.4 | 1.4 | 1.4 | 1.4 | 1.4 |
| Sulfate | | | | | | | | | | |
| Cocamidopropyl | 2 | 2 | 2 | 2 | 2 | 2 | 2 | 2 | 2 | 2 |
| Betaine | | | | | | | | | | |
| Rice bran oil | 0 | 0 | 0 | 0.5 | 0.5 | 0.5 | 1 | 1 | 1 | |
| PVOH 28-98⁽¹⁾ | 0 | 0.2 | 0.5 | 0 | 0.2 | 0.5 | 0 | 0.2 | 0.5 | |
| Guar | 0.4 | 0.4 | 0.4 | 0.4 | 0.4 | 0.4 | 0.4 | 0.4 | 0.4 | |
| Hydroxypropyl | | | | | | | | | | |
| Trimonium | | | | | | | | | | |
| Chloride ⁽²⁾ | | | | | | | | | | |
| Guar | | | | | | | | | | 0.4 |
| Hydroxypropyl | | | | | | | | | | |
| Trimonium | | | | | | | | | | |
| Chloride ⁽³⁾ | | | | | | | | | | |
| Gel Network | 2.3 | 2.3 | 2.3 | 2.3 | 2.3 | 2.3 | 2.3 | 2.3 | 2.3 | 1 |
| Premix from Table | | | | | | | | | | |
| 2 | | | | | | | | | | |
| Hydrogenated | 0.06 | 0.06 | 0.06 | 0.06 | 0.06 | 0.06 | 0.06 | 0.06 | 0.06 | |
| Castor Oil ⁽⁴⁾ | | | | | | | | | | |
| Ethylene Glycol | | | | | | | | | | 1.5 |
| Distearate ⁽⁵⁾ | | | | | | | | | | |
| Dimethicone ⁽⁶⁾ | | | | | | | | | | 3 |
| Citric Acid/ | Adjust to PH ~5-7 | | | | | | | | | |
| Sodium Citrate | | | | | | | | | | |
| Dihydrate/HCl/ | | | | | | | | | | |
| NAOH | | | | | | | | | | |

| | |
|---|---|
| Sodium Chloride/ | Adjust to viscosity of ~4-20 Pa · s |
| Ammonium | |
| Xylene Sulfonate | |
| Water, Fragrance, | Q.S. to 100 |
| Preservatives | |

Detangling force during combing, gf*

| | | | | | | | | | | |
|---|---|---|---|---|---|---|---|---|---|---|
| Detangling Wet | 535 | 423 | 351 | 497 | 297 | 321 | 417 | 321 | 299 | 312 |
| (Average) | | | | | | | | | | |
| Detangling Wet | 17 | 10 | 14 | 17 | 8 | 9 | 15 | 10 | 9 | 14 |
| (Std error) | | | | | | | | | | |
| Detangling | 417 | 321 | 299 | 178 | 144 | 130 | 162 | 138 | 119 | 147 |
| Dry(Average) | | | | | | | | | | |
| Detangling Dry | 15 | 10 | 9 | 14 | 10 | 5 | 10 | 12 | 5 | 7 |
| (Std error) | | | | | | | | | | |

*Values represent average gram force (gf) values for combing strokes 2 thru 6 (of a 10-stroke test), using a sample size of n = 3 hair tresses per product. Lower values represent less detangling force during combing and higher conditioning, which is preferred for the invention herein.
⁽¹⁾PVOH 28-98 (Kuraray Poval)
⁽²⁾ N-Hance 3196 (Ashland)
⁽³⁾ Jaguar ® C500 (Solvay)
⁽⁴⁾ Thixcin ® R (Elementis)
⁽⁵⁾ EGDS Purified (Evonik)
⁽⁶⁾ 330M Silicone (Momentive)

Table 2 shows the composition of the gel network premix in the Examples provided herein.

TABLE 2

| | (wt. %) |
|---|---|
| Sodium Laureth-1 Sulfate | 11.00 |
| Stearyl Alcohol | 7.71 |
| Cetyl Alcohol | 4.29 |
| Water | QS |

As can be seen in FIG. 1, for wet hair, the detangling results showed that Examples 1-4, each having greater than 0% of both PVOH and rice bran oil, performed similarly to or better than Comparative Examples having 0% of just rice bran oil (C2 and C3), Comparative Examples having 0% of just PVOH (C4 and C5), and Comparative Examples having 0% of both rice bran oil and PVOH (C1 and C6). This result is beneficial because C6 is the composition comprising silicone. Thus, the compositions comprising both botanical oil and PVOH performed comparably to a composition comprising silicone for wet detangling testing. Thus, from a consumer perspective, the compositions of Examples 1-4 and the composition of C6 perform consistently for wet detangling.

The results for dry detangling testing (FIG. 2) essentially mirror those for wet detangling testing. That is, the detangling force for the composition of Examples 1-4, each having greater than 0% of both PVOH and rice bran oil, was similar to or better than that for Comparative Examples having 0% of just rice bran oil (C2 and C3), Comparative Examples having 0% of just PVOH (C4 and C5), and Comparative Examples having 0% of both rice bran oil and PVOH (C1 and C6). Thus, the compositions comprising both botanical oil and PVOH performed comparably to a composition comprising silicone for dry detangling testing. Accordingly, from a consumer perspective, the compositions of Examples 1~4 and the composition of C6 perform consistently for dry detangling.

### Example 2: Fatty Alcohol Deposition Evaluation

The levels of fatty alcohol penetrating and depositing on the hair of five compositions was evaluated. Table 3 provides surface deposition and internal penetration data for Examples 5 and 6 and Comparative Examples C7, C8, and C9. The compositions for Examples 5 and 6 and C7, C8, and C9 were relatively similar. For example, all five compositions used the same amount of sodium laureth sulfate, sodium lauryl sulfate, cocamidoptopyl betaine, guar hydroxypropyl trimonium chloride, gel network premix, and hydrogenated castor oil. The exemplary compositions all also had botanical oil present but varied in the type and amount of the botanical oil. The exemplary compositions further differed in whether an amount of PVOH (PVOH 28-98) was present or not. In general, the Comparative Examples (C7-C9) did not have any PVOH and Examples 5 and 6 all contained both a combination of both an amount of a different type of botanical oil and an amount of PVOH.

Example 5 included 0.5% coconut oil and 0.2% PVOH. Example 6 included 0.5% jojoba oil and 0.5% PVOH. C7 included 0.75% shea butter and no PVOH. C8 included 1% rice bran oil and no PVOH. C9 included 0.5% rice bran oil and no PVOH.

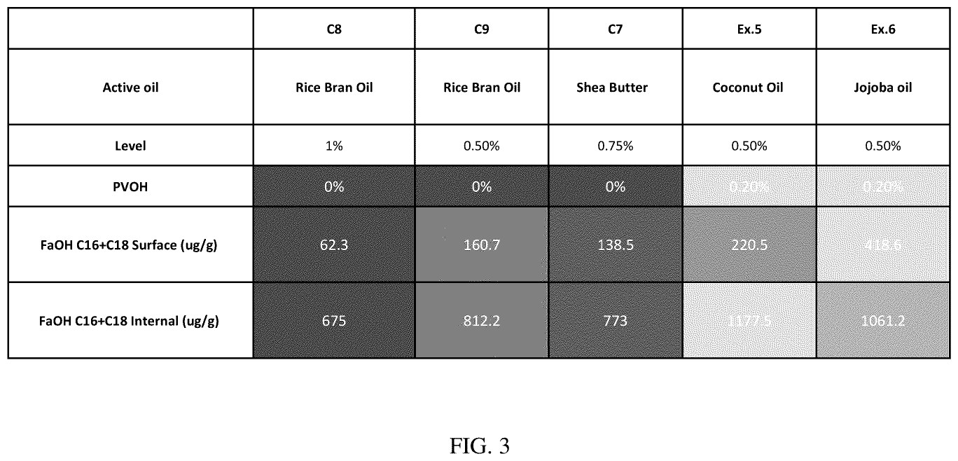

The fatty alcohol delivered to the hair, internally and on the surface, was measured according to the methods described herein. The delivery results are illustrated in FIG. 3.

TABLE 3

| | Ex. 5 | Ex. 6 | C7 | C8 | C9 |
|---|---|---|---|---|---|
| Sodium Laureth Sulfate | 11.6 | 11.6 | 11.6 | 11.6 | 11.6 |
| Sodium Lauryl Sulfate | 1.4 | 1.4 | 1.4 | 1.4 | 1.4 |
| Cocamidopropyl Betaine | 2 | 2 | 2 | 2 | 2 |
| Coconut oil | 0.5 | | | | |
| Jojoba oil | | 0.5 | | | |
| Shea butter | | | 0.75 | | |
| Rice bran oil | | | | 1 | 0.5 |
| Polyvinyl Alcohol | 0.2 | 0.2 | 0 | 0 | 0 |
| 28-98 ⁽¹⁾ | | | | | |
| Guar Hydroxypropyl | 0.4 | 0.4 | 0.4 | 0.4 | 0.4 |
| Trimonium Chloride ⁽²⁾ | | | | | |
| Gel Network Premix | 2.3 | 2.3 | 2.3 | 2.3 | 2.3 |
| from Table 2 | | | | | |
| Hydrogenated | 0.06 | 0.06 | 0.06 | 0.06 | 0.06 |
| Castor Oil ⁽³⁾ | | | | | |
| Citric Acid/Sodium | Adjust to PH ~5-7 | | | | |
| Citrate | | | | | |
| Dihydrate/HCl/NaOH | | | | | |
| Sodium Chloride/ | Adjust to viscosity of ~4-20 Pa · s | | | | |
| Ammonium Xylene | | | | | |
| Sulfonate | | | | | |
| Water, Fragrance, | Q.S. to 100 | | | | |
| Preservatives | | | | | |

Fatty Alcohol Deposition, ug/g *

| | | | | | |
|---|---|---|---|---|---|
| FaOH C16 + C18 | 220.5 | 418.6 | 138.5 | 62.3 | 160.7 |
| Surface (ug/g) | | | | | |
| FaOH C16 + C18 | 1177.5 | 1061.2 | 773 | 675 | 812.2 |
| Internal (ug/g) | | | | | |

* Values represent micrograms per gram (ug/g) concentration values on hair substrate, identified herein, after 5 shampoo cycles. Higher values represent more fatty alcohol delivered to the hair and higher conditioning, which is preferred for the invention herein. The composition for the gel network premix is provided in Table 2.
⁽¹⁾ PVOH 28-98 (Kuraray Poval)
⁽²⁾ N-Hance 3196 (Ashland)
⁽³⁾ Thixcin ® R (Elementis)

As can be seen in FIG. 3, the delivery results showed that the presence of PVOH in the composition leads to higher fatty alcohol deposition and penetration, as in Examples 5 and 6. In contrast, the Comparative Examples without PVOH (C7, C8, and C9) had lower amounts deposited and penetrating the hair. In FIG. 3, higher fatty alcohol delivery is indicated by lighter shading and lower fatty alcohol delivery is indicated by darker shading. Based on the above, from a consumer perspective, the compositions having both a botanical oil and PVOH, such as Examples 5 and 6, would provide desirable conditioning properties exceeding the benefit of botanical oils alone. Moreover, because Examples 5 and 6 comprise both botanical oil and PVOH (just as Examples 1-4 before did), the detangling effects of the compositions of Examples 5 and 6 will be acceptable for consumer use as well. In contrast, the compositions of C7-C9 comprise a botanical oil for conditioning but do not contain PVOH and would not obtain the synergistic conditioning effects, including the prior mentioned detangling benefits. Thus, compositions C7-C9 would not provide the conditioning benefits acceptable for consumer satisfaction.

### Example 3: Viscosity Evaluation

Aqueous conditioning shampoos that use botanical oils as conditioning agents often lose viscosity, becoming too watery for consumer preference. Traditional thickeners have proven ineffective at boosting viscosity without negatively affecting other consumer-desired properties.

In this example, in exemplary embodiments of conditioning shampoo compositions, coconut oil was used as a botanical oil conditioning agent and various grades of PVOH were added as 0.2 wt % relative to the total shampoo weight. In particular, the grades provided are of the format PVOH X-Y, where X refers to the viscosity grade which generally aligns with the viscosity in mPa-s of a specific concentration of PVOH, here 4% in water, at a standard temperature, here 20° C., and Y refers to a degree of hydrolysis. Thereby, A lower value for X generally represents PVOH of lower viscosity and a higher value represents PVOH of higher viscosity. The number provided as Y for the degree of hydrolysis generally indicates the percentage hydrolyzed. For example, in the instance Y is 80, the PVOH is 80% hydrolyzed and 20% of the acetate groups are left intact. Accordingly, PVOH 3-80 has lower viscosity and is less hydrolyzed than PVOH 56-98. This example demonstrates the synergistic impact of utilizing PVOH having viscosity grades greater than 20 and degrees of hydrolysis above 80% to help mitigate the negative impact of botanical oils on composition viscosity. Shampoo viscosity ranging from about 2000 mPa-s to about 20,000 mPa-s are considered suitable for consumer use.

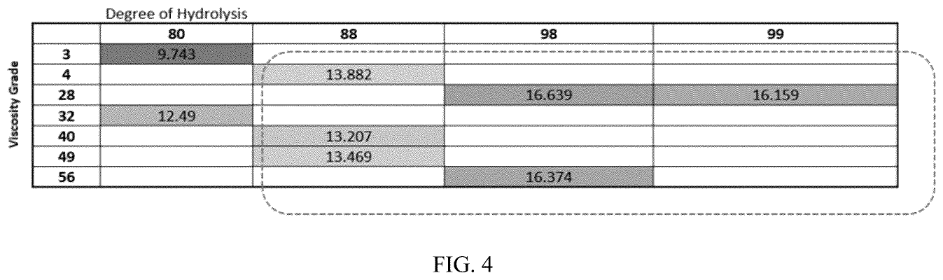

Table 4 provides composition and viscosity data for Examples 7-11 and Relative Examples R1, R2, and R3. Each of the Relative Examples utilizes grades of PVOH that are below at least one of the thresholds of a viscosity grade greater than 20 and a degree of hydrolysis above 80%. Examples 7-11 utilize grades of PVOH above both aforementioned thresholds. Viscosity was measured according to the methods described herein. The viscosity results are illustrated in FIG. 4.

TABLE 4

| | R1 | R2 | R3 | Ex. 7 | Ex. 8 | Ex. 9 | Ex. 10 | Ex. 11 |
|---|---|---|---|---|---|---|---|---|
| Sodium Laureth | 11.6 | 11.6 | 11.6 | 11.6 | 11.6 | 11.6 | 11.6 | 11.6 |
| Sulfate | | | | | | | | |
| Sodium Lauryl | 1.4 | 1.4 | 1.4 | 1.4 | 1.4 | 1.4 | 1.4 | 1.4 |
| Sulfate | | | | | | | | |
| Cocamidopropyl | 2 | 2 | 2 | 2 | 2 | 2 | 2 | 2 |
| Betaine | | | | | | | | |
| Coconut oil | 0.5 | 0.5 | 0.5 | 0.5 | 0.5 | 0.5 | 0.5 | 0.5 |
| Polyvinyl Alcohol | 0.2 | | | | | | | |
| 3-80 ⁽¹⁾ | | | | | | | | |
| Polyvinyl Alcohol | | 0.2 | | | | | | |
| 32-80 ⁽¹⁾ | | | | | | | | |
| Polyvinyl Alcohol | | | 0.2 | | | | | |
| 4-88 ⁽¹⁾ | | | | | | | | |
| Polyvinyl Alcohol | | | | 0.2 | | | | |
| 40-88 ⁽¹⁾ | | | | | | | | |
| Polyvinyl Alcohol | | | | | 0.2 | | | |
| 49-88 ⁽¹⁾ | | | | | | | | |
| Polyvinyl Alcohol | | | | | | 0.2 | | |
| 28-98 ⁽¹⁾ | | | | | | | | |
| Polyvinyl Alcohol | | | | | | | 0.2 | |
| 56-98 ⁽¹⁾ | | | | | | | | |
| Polyvinyl Alcohol | | | | | | | | 0.2 |
| 29-99 ⁽¹⁾ | | | | | | | | |
| Guar | 0.4 | 0.4 | 0.4 | 0.4 | 0.4 | 0.4 | 0.4 | 0.4 |
| Hydroxypropyl | | | | | | | | |
| Trimonium | | | | | | | | |
| Chloride ⁽²⁾ | | | | | | | | |
| Gel Network | 2.3 | 2.3 | 2.3 | 2.3 | 2.3 | 2.3 | 2.3 | 2.3 |
| Premix from Table 2 | | | | | | | | |
| Hydrogenated | 0.06 | 0.06 | 0.06 | 0.06 | 0.06 | 0.06 | 0.06 | 0.06 |
| Castor Oil ⁽³⁾ | | | | | | | | |
| Citric Acid/ | Adjust to PH ~5-7 | | | | | | | |
| Sodium Citrate | | | | | | | | |
| Dihydrate/HCl/ | | | | | | | | |
| NaOH | | | | | | | | |
| Water, Fragrance, | Q.S. to 100 | | | | | | | |
| Preservatives | | | | | | | | |
| Viscosity (Pa-s) | 9.74 | 12.49 | 13.88 | 13.2 | 13.469 | 16.64 | 16.37 | 16.16 |

⁽¹⁾ Polyvinyl Alcohol 3-80, 32-80, 4-88, 40-88, 49-88, 28-98, 56-98, 29-99 ((Kuraray Poval)
⁽²⁾ N-Hance 3196 (Ashland)
⁽³⁾ Thixcin ® R (Elementis)

FIG. 4 illustrates the viscosity of shampoo compositions for Relative Examples R1, R2, and R3 and Examples 7-11. Each composition contained 0.5% coconut oil and other common ingredients and amounts, including the 0.2% PVOH, and varied only in the respective grade of PVOH. Specifically, the composition of Relative Example R1 utilized PVOH 3-80, R2 utilized PVOH 32-80, and R³ utilized PVOH 4-88. Accordingly, all the compositions of the Relative Examples utilize PVOH which falls below one or both of the thresholds of being above 20 for viscosity grade and being above 80 for degree of hydrolysis.

Regarding the other compositions, the composition of Example 7 utilized PVOH 40-88, Example 8 utilized PVOH 49-88, Example 9 utilized PVOH 28-98, Example 10 utilized PVOH 56-98, and Example 11 utilized PVOH 29-99. Accordingly, all the compositions of the Examples 7-12 utilize PVOH above the thresholds provided above.

As shown in FIG. 4, higher viscosity grades and/or higher degrees of hydrolysis provide higher viscosity in resulting aqueous conditioning shampoo compositions. Indeed, the thresholds discussed above seem to provide for compositions with improved stability and ability to mitigate the negative impact of botanical oil on the viscosity of shampoo compositions having botanical oil. Indeed, the lowest measured viscosity was 9.74 Pa-s (9,740 mPa-s) for R1 and the highest measured viscosity was 16.64 Pa-s (16,640 mPa-s) for Example 9. All the Relative Examples, R1-R3, and all the Examples 7-11 were within the consumer-acceptable range of 2000 mPa-s to about 20,000 mPa-s. However, the higher degree of hydrolysis, and to a lesser degree, the higher viscosity grade, produced compositions with higher measured viscosities. This result shows that the compositions of Examples 7-11 had higher suitable viscosities, which emphasizes that ability for PVOH having grades above certain thresholds to produce aqueous conditioning shampoo compositions that can be used with various amounts of botanical oils to provide conditioning benefits and fatty alcohol delivery, while still having consumer-acceptable viscosities.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aqueous conditioning shampoo composition, comprising:
a) detersive surfactant;
b) botanical oil; and
c) about 0.02% to about 5% polyvinyl alcohol.

2. The aqueous conditioning shampoo composition of claim 1, wherein the botanical oil comprises triglycerides and esters of fatty acids, wherein the fatty acids are either saturated, monounsaturated, or polyunsaturated and wherein the fatty acids contain varying chain lengths ranging from C8 to C30.

3. The aqueous conditioning shampoo composition of claim 2, wherein the botanical oil is selected from soybean oil, canola oil, safflower oil, argan oil, jojoba oil, coconut oil, shea butter, orange peel wax, tea tree oil, and rice bran oil.

4. The aqueous conditioning shampoo composition of claim 1, wherein the composition comprises about 0.2 wt. % to about 5 wt. % of the botanical oil.

5. The aqueous conditioning shampoo composition of claim 1, wherein the composition is free of sulfated surfactants.

6. The aqueous conditioning shampoo composition of claim 1, further comprising a co-surfactant selected from alkyl amidopropylbetaine, alkyl betaine, alkanolamide, alkyl hydroxysultaine, an alpha olefin sulfonate, alkyl glucoside, and combinations thereof.

7. The aqueous conditioning shampoo composition of claim 1, wherein the composition is substantially free of silicone.

8. The aqueous conditioning shampoo composition of claim 1, wherein the composition has an average detangling value of less than 350 gf according to the Hair Texture Analysis Method.

9. An aqueous conditioning shampoo composition, comprising:
a) detersive surfactant;
b) botanical oil; and
c) polyvinyl alcohol, wherein the polyvinyl alcohol has at least one of the following: a) a degree of hydrolysis of greater than or equal to about 80% and b) a viscosity of greater than about 20 mPa·s, as measured as a 4% aqueous solution at 20° C. according to the PVOH Viscosity Method.

10. The aqueous conditioning shampoo composition of claim 9, wherein the botanical oil comprises triglycerides and esters of fatty acids, wherein the fatty acids are either saturated, monounsaturated, or polyunsaturated and wherein the fatty acids contain varying chain lengths ranging from C8 to C30.

11. The aqueous conditioning shampoo composition of claim 10, wherein the botanical oil is selected from soybean oil, canola oil, safflower oil, argan oil, jojoba oil, coconut oil, shea butter, orange peel wax, tea tree oil, and rice bran oil.

12. The aqueous conditioning shampoo composition of claim 9, wherein the composition comprises about 0.2 wt. % to about 5 wt. % of the botanical oil.

13. The aqueous conditioning shampoo composition of claim 9, wherein the composition comprises about 0.02 wt. % to about 5 wt. % of the polyvinyl alcohol.

14. The aqueous conditioning shampoo composition of claim 9, wherein the composition is free of sulfated surfactants and substantially free of silicone.

15. An aqueous conditioning shampoo composition, comprising:
a) detersive surfactant;
b) botanical oil; and
c) polyvinyl alcohol, wherein the composition has a viscosity of greater than about 2,000 mPa·s, as measured according to the Cone/Plate Viscosity Measurement Method.

16. The aqueous conditioning shampoo composition of claim 15, wherein the botanical oil comprises triglycerides and esters of fatty acids, wherein the fatty acids are either saturated, monounsaturated, or polyunsaturated and wherein the fatty acids contain varying chain lengths ranging from C8 to C30.

17. The aqueous conditioning shampoo composition of claim 16, wherein the botanical oil is selected from soybean oil, canola oil, safflower oil, argan oil, jojoba oil, coconut oil, shea butter, orange peel wax, tea tree oil, and rice bran oil.

18. The aqueous conditioning shampoo composition of claim 15, wherein the composition comprises about 0.2 wt. % to about 5 wt. % of the botanical oil.

19. The aqueous conditioning shampoo composition of claim 15, wherein the comprises about 0.02 wt. % to about 1 wt. % of the polyvinyl alcohol.

20. The aqueous conditioning shampoo composition of claim 15, wherein the composition is free of sulfated surfactants and substantially free of silicone.
